(19) United States
(12) Patent Application Publication
(10) Pub. No.: US 2005/0234068 A1
(43) Pub. Date: Oct. 20, 2005

(54) Composition and method of decreasing renal ischemic damage

(76) Inventor: Dalton D. Baldwin, Loma Linda, CA (US)

Correspondence Address: David A. Farah, M.D.;SHELDON & MAK PC, Suite 900, 225 South Lake Avenue, Pasadena, CA, 91101, US

(21) Appl. No.: 11/108,917

(22) Filed: Apr. 18, 2005

Related U.S. Application Data
(60) Provisional application No. 60/563,772, filed on Apr. 19, 2004.
(60) Provisional application No. 60/578,531, filed on Jun. 9, 2004.

(51) Int. Cl. A61K 31/519

(52) U.S. Cl. 514/252.16; 514/262.1

(57) ABSTRACT

A method of decreasing renal ischemic damage comprising a) identifying an organism having a kidney that is susceptible to renal ischemic damage from an ischemic event; and b) administering to the organism one or more than one effective dose of an agent prior to the ischemic event; where administering to the organism the one or more than one effective dose of the agent serves to at least partially protect the organism's kidney from damage during a subsequent ischemic event. A composition for decreasing renal ischemic damage comprising one or more than one phosphodiesterase inhibitor, and one or more than one HMG-CoA reductase inhibitor.

### CROSS-REFERENCE TO RELATED APPLICATIONS

The present Application claims the benefit of U.S. Provisional Patent Application 60/563,772, titled “Composition and Method of Decreasing Renal Ischemic Damage” filed Apr. 19, 2004; and claims the benefit of U.S. Provisional Patent Application 60/578,531, titled “Composition and Method of Decreasing Renal Ischemic Damage” filed Jun. 9, 2004; the contents of which are incorporated in this disclosure by reference in their entirety.

### BACKGROUND

There are a variety of diseases and conditions that cause ischemia of the kidney, leading to kidney dysfunction. These diseases and conditions include partial nephrectomy for tumor and congenital anomalies, renal trauma, iatrogenic renal injuries and renal vascular surgery. Further, kidney removal for transplantation is also associated with ischemic damage. In the United States alone, there are approximately 9,000 kidney transplants each year. Of these, approximately 500 are found to have significant ischemic damage during transplantation. There is, at present, no practical and effective method known to decrease renal ischemic damage associated with these diseases and conditions.

The mechanism of renal ischemic damage is partly understood. Animal studies have demonstrated that deliberately decreasing renal blood flow and glomerular filtration rate using CO₂ pneumoperitoneum preconditioning prior to the ischemic event significantly decreases the area and amount of ischemic damage. The effect of such limited ischemic preconditioning appears to lead to an increase in nitric oxide metabolites due to an increase in endothelial nitric oxide synthase.

Disadvantageously, however, deliberately causing limited ischemia prior to an ischemic event is not a practical method when preventing renal ischemic damage in the context of a live kidney donor, among other circumstances. Therefore, there is a need for another method of decreasing renal ischemic damage in an organism, including a human.

### SUMMARY

According to one embodiment of the present invention, there is provided a method of decreasing renal ischemic damage. The method comprises, first, identifying an organism having a kidney that is susceptible to renal ischemic damage from an ischemic event, and second, administering to the organism one or more than one effective dose of an agent prior to the ischemic event; where administering to the organism the one or more than one effective dose of the agent serves to at least partially protect the organism's kidney from damage during a subsequent ischemic event. In one embodiment, the ischemic event is removal of the kidney for transplantation into another organism.

In one embodiment, the agent is a phosphodiesterase inhibitor. In another embodiment, the agent is a composition comprising a phosphodiesterase inhibitor. In one embodiment, the phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor. In another embodiment, the phosphodiesterase inhibitor is a type 5 phosphodiesterase inhibitor. In another embodiment, the phosphodiesterase inhibitor is selected from the group consisting of sildenafil, tadalafil and vardenafil.

In one embodiment, the agent is an HMG-CoA reductase inhibitor. In another embodiment, the agent is a composition comprising an HMG-CoA reductase inhibitor. In one embodiment, the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

In one embodiment, the agent is a composition comprising both one or more than one phosphodiesterase inhibitor and one or more than one HMG-CoA reductase inhibitor. In one embodiment, the phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor. In another embodiment, the phosphodiesterase inhibitor is a type 5 phosphodiesterase inhibitor. In another embodiment, the phosphodiesterase inhibitor is selected from the group consisting of sildenafil, tadalafil and vardenafil. In one embodiment, the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

In one embodiment, the dose of the agent is between about 1 mg and 1 g. In another embodiment, the dose of the agent is between about 10 mg and 200 mg. In another embodiment, the dose of the agent is between about 25 mg to 100 mg.

In one embodiment, the organism has a body weight and the dose of the agent is between about 0.015 mg/kg body weight and 15 mg/kg body weight. In another embodiment, the organism has a body weight and the dose of the agent is between about 0.15 mg/kg body weight and 3 mg/kg body weight. In another embodiment, the organism has a body weight and the dose of the agent is between about 0.5 mg/kg body weight and 1.5 mg/kg body weight.

In one embodiment, the agent is administered orally. In another embodiment, the agent is administered by a route selected from the group consisting of administration by skin patch, administration by subcutaneous injection, administration by an inhaled preparation and direct intravenous administration.

In one embodiment, the one or more than one dose of the agent is between about 1 dose and 10 doses. In another embodiment, the one or more than one dose of the agent is between about 2 doses and 6 doses. In another embodiment, the one or more than one dose of the agent is three doses.

In one embodiment, the one or more than one dose is a plurality of doses administered between about 1 minute and 2 days apart. In another embodiment, the one or more than one dose is a plurality of doses administered between about 10 minutes and 1 day apart. In another embodiment, the one or more than one dose is a plurality of doses administered between about 30 minutes and 4 hours apart.

According to another embodiment of the present invention, there is provided a composition for decreasing renal ischemic damage comprising two or more than two phosphodiesterase inhibitors. In one embodiment, the two or more than two phosphodiesterase inhibitors are selected from the group consisting of a type 3 phosphodiesterase inhibitor, a type 4 phosphodiesterase inhibitor, a type 5 phosphodiesterase inhibitor and a type 9 phosphodiesterase inhibitor. In another embodiment, at least one of the two or more than two phosphodiesterase inhibitors is a type 5 phosphodiesterase inhibitor. In one embodiment, at least one of the two or more than two phosphodiesterase inhibitors is selected from the group consisting of sildenafil, tadalafil and vardenafil. In one embodiment, the amount of at least one of the two or more than two phosphodiesterase inhibitors is between about 1 mg and 1 g. In another embodiment, the amount of at least one of the two or more than two phosphodiesterase inhibitors is between about 10 mg and 200 mg. In another embodiment, the amount of at least one of the two or more than two phosphodiesterase inhibitors is between about 25 mg to 100 mg. In another embodiment, the composition further comprises one or more than one substance selected from the group consisting of a binding agent, a coloring agent, an enteric coating and a flavoring agent.

According to another embodiment of the present invention, there is provided a composition for decreasing renal ischemic damage comprising two or more than two HMG-CoA reductase inhibitors. In one embodiment, at least one of the two or more than two HMG-CoA reductase inhibitors is selected from the group consisting of atorvastatin calcium, fluvastatin sodium, lovastatin, pitavastatin calcium, pravastatin sodium, rosuvastatin calcium and simvastatin. In one embodiment, the amount of at least one of the two or more than two HMG-CoA reductase inhibitors is between about 1 mg and 1 g. In another embodiment, the amount of at least one of the two or more than two HMG-CoA reductase inhibitors is between about 10 mg and 200 mg. In another embodiment, the amount of at least one of the two or more than two HMG-CoA reductase inhibitors is between about 25 mg to 100 mg. In another embodiment, the composition further comprises one or more than one substance selected from the group consisting of a binding agent, a coloring agent, an enteric coating and a flavoring agent.

According to another embodiment of the present invention, there is provided a composition for decreasing renal ischemic damage comprising one or more than one phosphodiesterase inhibitor, and one or more than one HMG-CoA reductase inhibitor. In one embodiment, at least one of the one or more than one phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor. In another embodiment, at least one of the one or more than one phosphodiesterase inhibitor is a type 5 phosphodiesterase inhibitor. In another embodiment, at least one of the one or more than one phosphodiesterase inhibitor is selected from the group consisting of sildenafil, tadalafil and vardenafil. In another embodiment, at least one of the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin. In one embodiment, the amount of at least one of the one or more than one phosphodiesterase inhibitor is between about 1 mg and 1 g. In another embodiment, the amount of at least one of the one or more than one phosphodiesterase inhibitor is between about 10 mg and 200 mg. In another embodiment, the amount of at least one of the one or more than one phosphodiesterase inhibitor is between about 25 mg to 100 mg. In one embodiment, the amount of at least one of the one or more than one HMG-CoA reductase inhibitor is between about 1 mg and 1 g. In another embodiment, the amount of at least one of the one or-more than one HMG-CoA reductase inhibitor is between about 10 mg and 200 mg. In another embodiment, the amount of at least one of the one or more than one HMG-CoA reductase inhibitor is between about 25 mg to 100 mg. In another embodiment, the composition further comprises one or more than one substance selected from the group consisting of a binding agent, a coloring agent, an enteric coating and a flavoring agent.

### DESCRIPTION

According to one embodiment of the present invention, there is provided a method of decreasing renal ischemic damage in an organism, including a human, including decreasing renal ischemic damage in an organism serving as a kidney donor during renal transplantation. The method comprises administering to the organism one or more than one effective dose of one or more than one phosphodiesterase inhibitor, or one or more than one HMG-CoA reductase inhibitor, or a combination of one or more phosphodiesterase inhibitor and one or more than one HMG-CoA reductase inhibitor. In a preferred embodiment, the method comprises administering to the organism one or more than one effective dose of a composition according to the present invention. According to another embodiment of the present invention, there is provided a composition for decreasing renal ischemic damage. The method and composition will now be disclosed in detail.

As used in this disclosure, the term “comprise” and variations of the term, such as “comprising” and “comprises,” are not intended to exclude other additives, components, integers or steps.

As used in this disclosure, the term “organism” includes a human.

According to one embodiment of the present invention, there is provided a method of decreasing renal ischemic damage in an organism subjected to an ischemic event. In one embodiment, the organism is serving as a kidney donor during renal transplantation, and the ischemic event is removal of the kidney for transplantation into another organism. The method comprises, first, identifying an organism having a kidney that is susceptible to renal ischemic damage from an ischemic event. Next, the method comprises administering to the organism one or more than one effective dose of an agent prior to the ischemic event. Administering to the organism the one or more than one effective dose of the agent serves to at least partially protect the organism's kidney from damage during a subsequent ischemic event.

In one embodiment, the agent is a phosphodiesterase inhibitor. In a preferred embodiment, the agent is a composition comprising a phosphodiesterase inhibitor. In a preferred embodiment, the phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor. In a particularly preferred embodiment, the phosphodiesterase inhibitor is a type 5 phosphodiesterase inhibitor.

In another embodiment the agent is an HMG-CoA reductase inhibitor. In a preferred embodiment, the agent is a composition comprising an HMG-CoA reductase inhibitor. In one embodiment, the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

In a preferred embodiment, the agent is a composition comprising both one or more than one phosphodiesterase inhibitor and one or more than one HMG-CoA reductase inhibitor. In a preferred embodiment, the one or more than one phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor. In a particularly preferred embodiment, the phosphodiesterase inhibitor is a type 5 phosphodiesterase inhibitor.

Though not intending to be limited to any particular theory, the phosphodiesterase inhibitor appears to protect the organism's kidney from a subsequent ischemic event by increasing nitric oxide levels within the organism, thereby mimicking the action of limited ischemic preconditioning. The HMG-CoA reductase inhibitors appear to protect the organism's kidney from a subsequent ischemic event by acting as a nitric oxide donor or through the HMG-CoA reductase inhibition pathway.

In one embodiment, the phosphodiesterase inhibitor is selected from the group consisting of sildenafil (Viagra®, available from Pfizer, Inc., New York, N.Y. US), tadalafil (Cialis®, available from Lilly ICOS, L.L.C. Delaware, Md. US) and vardenafil (Levitra®, available from Bayer Aktiengesellschaft, Germany), though other phosphodiesterase inhibitors can be used, as will be understood by those with skill in the art with reference to this disclosure. In another embodiment, the HMG-CoA reductase inhibitor is selected from the group consisting of atorvastatin calcium (Lipitor®, available from Pfizer, Inc., New York, N.Y. US), fluvastatin sodium (Lescol® available from Novartis AG Corporation, Basel, Switzerland), lovastatin (Mevacor® available from Merck & Co., Inc., N.J. US), pitavastatin calcium (Livalo available from Kowa Company Ltd., Naka-ku Nagoya JP), pravastatin sodium (Pravachol® available from Bristol-Myers Squibb Company, New York, N.Y. US), simvastatin (Zocor® available from Merck & Co., Inc., Whitehouse Station, N.J. US), and rosuvastatin calcium (Crestor®, available from IPR Pharmaceuticals Inc., Puerto Rico US), though other HMG-CoA reductase inhibitors can be used, as will be understood by those with skill in the art with reference to this disclosure.

In one embodiment, the dose of the agent for an adult human is between about 1 mg and 1 g. In another embodiment, the dose of the agent for an adult human is between about 10 mg and 200 mg. In another embodiment, the dose of the agent for an adult human is between about 25 mg to 100 mg.

In one embodiment, the organism has a body weight and the dose of the agent is between about 0.015 mg/kg body weight and 15 mg/kg body weight. In another embodiment, the organism has a body weight and the dose of the agent between about 0.15 mg/kg body weight and 3 mg/kg body weight. In another embodiment, the organism has a body weight and the dose of the agent is between about 0.5 mg/kg body weight and 1.5 mg/kg body weight.

In a preferred embodiment, the agent is administered orally, though other routes of administration are also within the scope of this invention, as will be understood by those with skill in the art with reference to this disclosure including administration by a route selected from the group consisting of administration by skin patch, administration by subcutaneous injection, administration by an inhaled preparation and direct intravenous administration.

In a preferred embodiment, the one or more than one dose of the agent is between about 1 dose and 10 doses. In another embodiment, the one or more than one dose of the agent is between about 2 doses and 6 doses. In another embodiment, the one or more than one dose of the agent is three doses.

In a preferred embodiment, the one or more than one dose is a plurality of doses administered between about 1 minute and 2 days apart. In another preferred embodiment, the one or more than one dose is a plurality of doses administered between about 10 minutes and 1 day apart. In another preferred embodiment, the one or more than one dose is a plurality of doses administered between about 30 minutes and 4 hours apart.

According to another embodiment of the present invention, there is provided a composition for decreasing renal ischemic damage. In one embodiment, the composition comprises two or more than two phosphodiesterase inhibitors. In a preferred embodiment, the phosphodiesterase inhibitors are selected from the group consisting of a type 3 phosphodiesterase inhibitor, a type 4 phosphodiesterase inhibitor, a type 5 phosphodiesterase inhibitor and a type 9 phosphodiesterase inhibitor. In a preferred embodiment, the phosphodiesterase inhibitor is selected from the group consisting of sildenafil, tadalafil and vardenafil. In another embodiment, the composition comprises two or more than two HMG-CoA reductase inhibitors. In a preferred embodiment, the HMG-CoA reductase inhibitors are selected from the group consisting of atorvastatin calcium, fluvastatin sodium, lovastatin, pitavastatin calcium, pravastatin sodium, rosuvastatin calcium and simvastatin. In another embodiment, the composition comprises two or more than two agents selected from the group consisting of sildenafil, tadalafil, vardenafil, atorvastatin calcium, fluvastatin sodium, lovastatin, pitavastatin calcium, pravastatin sodium, rosuvastatin calcium and simvastatin.

In one embodiment, the amount of each agent in the composition is between about 1 mg and 1 g. In another embodiment, the amount of each agent in the composition is between about 10 mg and 200 mg. In another embodiment, the amount of each agent in the composition is between about 25 mg to 100 mg.

As will be understood by those with skill in the art with reference to this disclosure, the composition of the present invention can also comprise one or more than one additional substance, such a binding agent, a coloring agent, an enteric coating and a flavoring agent. The composition is preferably configured to be administered orally, however, it can also be configured to be administered by skin patch, subcutaneous injection, inhaled preparations or direct intravenous administration, among other routes, as will be understood by those with skill in the art with reference to this disclosure.

### EXAMPLE I

### Method of Decreasing Renal Ischemic Damage During Transplantation

The method of the present invention can be used as follows. Patient 1, a 60-kg male, is a suitable live donor for a kidney for patient 2, also a 60-kg male and a dialysis patient having no significant residual natural kidney function. Patient 1 is administered three 100 mg doses of sildenafil orally, once a day for 3 days prior to the surgery to remove the donated kidney, thereby decreasing the risk of renal ischemic damage to the donated kidney. The donated kidney is then harvested and implanted in patient 2.

Although the present invention has been discussed in considerable detail with reference to certain preferred embodiments, other embodiments are possible. Therefore, the scope of the appended claims should not be limited to the description of preferred embodiments contained herein.

1. A method of decreasing renal ischemic damage comprising:
a) identifying an organism having a kidney that is susceptible to renal ischemic damage from an ischemic event; and
b) administering to the organism one or more than one effective dose of an agent prior to the ischemic event;
where administering to the organism the one or more than one effective dose of the agent serves to at least partially protect the kidney from damage during a subsequent ischemic event.

2. The method of claim 1, where the ischemic event is removal of the kidney for transplantation into another organism.

3. The method of claim 1, where the agent is a phosphodiesterase inhibitor.

4. The method of claim 1, where the agent is a composition comprising a phosphodiesterase inhibitor.

5. The method of claim 3, where the agent is a phosphodiesterase inhibitor selected from the group consisting of a type 3 phosphodiesterase inhibitor, a type 4 phosphodiesterase inhibitor, a type 5 phosphodiesterase inhibitor and a type 9 phosphodiesterase inhibitor.

6. The method of claim 3, where the agent is a type 5 phosphodiesterase inhibitor.

7. The method of claim 3, where the phosphodiesterase inhibitor is selected from the group consisting of sildenafil, tadalafil and vardenafil.

8. The method of claim 4, where the agent is a phosphodiesterase inhibitor selected from the group consisting of a type 3 phosphodiesterase inhibitor, a type 4 phosphodiesterase inhibitor, a type 5 phosphodiesterase inhibitor and a type 9 phosphodiesterase inhibitor.

9. The method of claim 4, where the agent is a type 5 phosphodiesterase inhibitor.

10. The method of claim 4, where the phosphodiesterase inhibitor is selected from the group consisting of sildenafil, tadalafil and vardenafil.

11. The method of claim 1, where the agent is one or more than one HMG-CoA reductase inhibitor.

12. The method of claim 1, where the agent is a composition comprising one or more than one HMG-CoA reductase inhibitor.

13. The method of claim 11, where the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

14. The method of claim 12, where the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

15. The method of claim 1, where the agent is a composition comprising both one or more than one phosphodiesterase inhibitor and one or more than one HMG-CoA reductase inhibitor.

16. The method of claim 15, where the one or more than one phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor.

17. The method of claim 16, where the one or more than one phosphodiesterase inhibitor is a type 5 phosphodiesterase inhibitor.

18. The method of claim 16, the one or more than one phosphodiesterase inhibitor is selected from the group consisting of sildenafil, tadalafil and vardenafil.

19. The method of claim 16, the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

20. The method of claim 1, where the dose of the agent is between about 1 mg and 1 g.

21. The method of claim 1, where the dose of the agent is between about 10 mg and 200 mg.

22. The method of claim 1, where the dose of the agent is between about 25 mg to 100 mg.

23. The method of claim 1, where the organism has a body weight and the dose of the agent is between about 0.015 mg/kg body weight and 15 mg/kg body weight.

24. The method of claim 1, where the organism has a body weight and the dose of the agent is between about 0.15 mg/kg body weight and 3 mg/kg body weight.

25. The method of claim 1, where the organism has a body weight and the dose of the agent is between about 0.5 mg/kg body weight and 1.5 mg/kg body weight.

26. The method of claim 1, where the agent is administered orally.

27. The method of claim 1, where the agent is administered by a route selected from the group consisting of administration by skin patch, administration by subcutaneous injection, administration by an inhaled preparation and direct intravenous administration.

28. The method of claim 1, where the one or more than one dose of the agent is between about 1 dose and 10 doses.

29. The method of claim 1, where the one or more than one dose of the agent is between about 2 doses and 6 doses.

30. The method of claim 1, where the one or more than one dose of the agent is three doses.

31. The method of claim 1, where the one or more than one dose is a plurality of doses administered between about 1 minute and 2 days apart.

32. The method of claim 1, where the one or more than one dose is a plurality of doses administered between about 10 minutes and a 1 day apart.

33. The method of claim 1, where the one or more than one dose is a plurality of doses administered between about 30 minutes and 4 hours apart.

34. A composition for decreasing renal ischemic damage comprising two or more than two phosphodiesterase inhibitors.

35. The composition of claim 34, where the two or more than two phosphodiesterase inhibitors are selected from the group consisting of a type 3 phosphodiesterase inhibitor, a type 4 phosphodiesterase inhibitor, a type 5 phosphodiesterase inhibitor and a type 9 phosphodiesterase inhibitor.

36. The composition of claim 34, where at least one of the two or more than two phosphodiesterase inhibitors is a type 5 phosphodiesterase inhibitor.

37. The composition of claim 34, where at least one of the two or more than two phosphodiesterase inhibitors is selected from the group consisting of sildenafil, tadalafil and vardenafil.

38. The composition of claim 34, where the amount of at least one of the two or more than two phosphodiesterase inhibitors is between about 1 mg and 1 g.

39. The composition of claim 34, where the amount of at least one of the two or more than two phosphodiesterase inhibitors is between about 10 mg and 200 mg.

40. The composition of claim 34, where the amount of at least one of the two or more than two phosphodiesterase inhibitors is between about 25 mg to 100 mg.

41. The composition of claim 34, further comprising one or more than one substance selected from the group consisting of a binding agent, a coloring agent, an enteric coating and a flavoring agent.

42. A composition for decreasing renal ischemic damage comprising two or more than two HMG-CoA reductase inhibitors.

43. The composition of claim 42, where at least one of the two or more than two HMG-CoA reductase inhibitors is selected from the group consisting of atorvastatin calcium, fluvastatin sodium, lovastatin, pitavastatin calcium, pravastatin sodium, rosuvastatin calcium and simvastatin.

44. The composition of claim 42, where the amount of at least one of the two or more than two phosphodiesterase inhibitors is between about 1 mg and 1 g.

45. The composition of claim 42, where the amount of at least one of the two or more than two phosphodiesterase inhibitors is between about 10 mg and 200 mg.

46. The composition of claim 42, where the amount of at least one of the two or more than two phosphodiesterase inhibitors is between about 25 mg to 100 mg.

47. The composition of claim 42, further comprising one or more than one substance selected from the group consisting of a binding agent, a coloring agent, an enteric coating and a flavoring agent.

48. A composition for decreasing renal ischemic damage comprising one or more than one phosphodiesterase inhibitor, and one or more than one HMG-CoA reductase inhibitor.

49. The composition of claim 48, where at least one of the one or more than one phosphodiesterase inhibitor is selected from the group consisting of a type 3 phosphodiesterase inhibitor, a type 4 phosphodiesterase inhibitor, a type 5 phosphodiesterase inhibitor and a type 9 phosphodiesterase inhibitor.

50. The composition of claim 48, where at least one of the one or more than one phosphodiesterase inhibitor is a type 5 phosphodiesterase inhibitor.

51. The composition of claim 48, where at least one of the one or more than one phosphodiesterase inhibitor is selected from the group consisting of sildenafil, tadalafil and vardenafil.

52. The composition of claim 48, where at least one of the one or more than one HMG-CoA reductase inhibitor is selected from the group consisting of atorvastatin calcium, fluvastatin sodium, lovastatin, pitavastatin calcium, pravastatin sodium, rosuvastatin calcium and simvastatin.

53. The composition of claim 48, where the amount of at least one of the one or more than one phosphodiesterase inhibitor is between about 1 mg and 1 g.

54. The composition of claim 48, where the amount of at least one of the one or more than one phosphodiesterase inhibitor is between about 10 mg and 200 mg.

55. The composition of claim 48, where the amount of at least one of the one or more than one phosphodiesterase inhibitor is between about 25 mg to 100 mg.

56. The composition of claim 48, where the amount of at least one of the one or more than one HMG-CoA reductase inhibitor is between about 1 mg and 1 g.

57. The composition of claim 48, where the amount of at least one of the one or more than one HMG-CoA reductase inhibitor is between about 10 mg and 200 mg.

58. The composition of claim 48, where the amount of at least one of the one or more than one HMG-CoA reductase inhibitor is between about 25 mg to 100 mg.

59. The composition of claim 48, further comprising one or more than one substance selected from the group consisting of a binding agent, a coloring agent, an enteric coating and a flavoring agent.
